SEQUENCE LISTING (20 sequences)

SEQ ID NO: 1 - protein, 214 aa, Bos taurus
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 2 - protein, 178 aa, Bos taurus
MKCLLLALALTCGAQALIVTQTMKGLDIQKVAGTWYSLAMAASDISLLDAQSAPLRVYVEELKPTPEGDLEILLQKWENDECAQKKIIAEKTKIPAVFKIDALNENKVLVLDTDYKKYLLVCMENSAEPEQSLVCQCLVRTPEVDDEALEKFDKALKALPMHIRLSFNPTQLEEQCHI

SEQ ID NO: 3 - protein, 459 aa, Pisum sativum
MAATTMKASFPLLMLMGISFLASVCVSSRSDPQNPFIFKSNKFQTLFENENGHIRLLQKFDQRSKIFENLQNYRLLEYKSKPHTIFLPQHTDADYILVVLSGKAILTVLKPDDRNSFNLERGDTIKLPAGTIAYLVNRDDNEELRVLDLAIPVNRPGQLQSFLLSGNQNQQNYLSGFSKNILEASFNTDYEEIEKVLLEEHEKETQHRRSLKDKRQQSQEENVIVKLSRGQIEELSKNAKSTSKKSVSSESEPFNLRSRGPIYSNEFGKFFEITPEKNPQLQDLDIFVNSVEIKEGSLLLPHYNSRAIVIVTVNEGKGDFELVGQRNENQQEQRKEDDEEEEQGEEEINKQVQNYKAKLSSGDVFVIPAGHPVAVKASSNLDLLGFGINAENNQRNFLAGDEDNVISQIQRPVKELAFPGSAQEVDRILENQKQSHFADAQPQQRERGSRETRDRLSSV

SEQ ID NO: 4 - protein, 496 aa, Oryza sativa
MASINRPIVFFTVCLFLLCNGSLAQQLLGQSTSQWQSSRRGSPRECRFDRLQAFEPIRSVRSQAGTTEFFDVSNEQFQCTGVSVVRRVIEPRGLLLPHYTNGASLVYIIQGRGITGPTFPGCPESYQQQFQQSGQAQLTESQSQSQKFKDEHQKIHRFRQGDVIALPAGVAHWCYNDGEVPVVAIYVTDLNNGANQLDPRQRDFLLAGNKRNPQAYRREVEERSQNIFSGFSTELLSEALGVSGQVARQLQCQNDQRGEIVRVEHGLSLLQPYASLQEQEQGQVQSRERYQEGQYQQSQYGSGCSNGLDETFCTLRVRQNIDNPNRADTYNPRAGRVTNLNTQNFPILSLVQMSAVKVNLYQNALLSPFWNINAHSVVYITQGRARVQVVNNNGKTVFNGELRRGQLLIIPQHYAVVKKAQREGCAYIAFKTNPNSMVSHIAGKSSIFRALPNDVLANAYRISREEAQRLKHNRGDEFGAFTPIQYKSYQDVYNAA

SEQ ID NO: 5 - protein, 495 aa, Glycine max
MAKLVFSLCFLLFSGCCFAFSSREQPQQNECQIQKLNALKPDNRIESEGGLIETWNPNNKPFQCAGVALSRCTLNRNALRRPSYTNGPQEIYIQQGKGIFGMIYPGCPSTFEEPQQPQQRGQSSRPQDRHQKIYNFREGDLIAVPTGVAWWMYNNEDTPVVAVSIIDTNSLENQLDQMPRRFYLAGNQEQEFLKYQQEQGGHQSQKGKHQQEEENEGGSILSGFTLEFLEHAFSVDKQIAKNLQGENEGEDKGAIVTVKGGLSVIKPPTDEQQQRPQEEEEEEEDEKPQCKGKDKHCQRPRGSQSKSRRNGIDETICTMRLRHNIGQTSSPDIYNPQAGSVTTATSLDFPALSWLRLSAEFGSLRKNAMFVPHYNLNANSIIYALNGRALIQVVNCNGERVFDGELQEGRVLIVPQNFVVAARSQSDNFEYVSFKTNDTPMIGTLAGANSLLNALPEEVIQHTFNLKSQQARQIKNNNPFKFLVPPQESQKRAVA

SEQ ID NO: 6 - protein, 12 aa, Bos taurus
FFVAPFPEVFGK

SEQ ID NO: 7 - protein, 10 aa, Bos taurus
YLGYLEQLLR

SEQ ID NO: 8 - protein, 15 aa, Bos taurus
HQGLPQEVLNENLLR

SEQ ID NO: 9 - protein, 20 aa, Bos taurus
VYVEELKPTPEGDLEILLQK

SEQ ID NO: 10 - protein, 9 aa, Bos taurus
VLVLDTDYK

SEQ ID NO: 11 - protein, 8 aa, Bos Taurus
LIVTQTMK

SEQ ID NO: 12 - protein, 12 aa, Pisum sativum
EGSLLLPHYNSR

SEQ ID NO: 13 - protein, 9 aa, Pisum sativum
GDFELVGQR

SEQ ID NO: 14 - protein, 10 aa, Pisum sativum
GPIYSNEFGK

SEQ ID NO: 15 - protein, 12 aa, Oryza sativa
ALPNDVLANAYR

SEQ ID NO: 16 - protein, 8 aa, Oryza sativa
LQAFEPIR

SEQ ID NO: 17 - protein, 13 aa, Oryza sativa
GDEFGAFTPIQYK

SEQ ID NO: 18 - protein, 13 aa, Glycine max
VLIVPQNFVVAAR

SEQ ID NO: 19 - protein, 10 aa, Glycine max
VFDGELQEGR

SEQ ID NO: 20 - protein, 9 aa, Glycine max
LNALKPDNR

(19) United States
(12) Patent Application Publication
(10) Pub. No.: US 2018/0011107 A1
(43) Pub. Date: Jan. 11, 2018

(54) IDENTIFICATION OF THE PRESENCE OF SPECIFIC POLYPEPTIDES BY LIQUID CHROMATOGRAPHY AND MASS SPECTROMETRY

(71) Applicant: Genysis Lab Inc., Salt Lake City, UT (US)

(72) Inventors: Michael Landesman, Salt Lake City, UT (US); Spencer Carter, Salt Lake City, UT (US)

(73) Assignee: Genysis Lab Inc., Salt Lake City, UT (US)

(21) Appl. No.: 15/641,907

(22) Filed: Jul. 5, 2017

Related U.S. Application Data
(60) Provisional application No. 62/359,310, filed on Jul. 7, 2016.

(51) Int. Cl. G01N 33/68; C12P 21/06

(52) U.S. Cl. CPC G01N 33/6848; C12Y 304/21004; C12P 21/06; G01N 2570/00; G01N 2560/00

(57) ABSTRACT

Disclosed are methods for determining the presence of one or more proteins in a sample, the methods comprising: enzymatically digesting the sample with a protease activity to generate a plurality of proteolytic peptides; separating the plurality of proteolytic peptides using liquid chromatography; performing mass spectrometry on the separated plurality of peptides; and wherein a protein is present in the sample when three or more target peptides for the protein are present among the proteolytic peptides; and wherein the target peptides are selected from the groups consisting of SEQ ID NOS:6-8, SEQ ID NOS:9-11, SEQ ID NOS:12-14, SEQ ID NOS:15-17, and SEQ ID NOS:18-20. In embodiments, a known quantity of a standard peptide may be added to the proteolytic peptides.

### CROSS-REFERENCE TO RELATED APPLICATION

This application claims the benefit under 35 U.S.C. §119(e) of U.S. Provisional Patent Application Ser. No. 62/359,310, filed Jul. 7, 2016, the disclosure of which is hereby incorporated herein in its entirety by this reference.

### TECHNICAL FIELD

The present application relates generally to biotechnology. More specifically, embodiments of the application relate to the detection of specific proteins in a sample through the use of liquid chromatography and mass spectrometry.

### BACKGROUND

In various fields ranging from fundamental biology to clinical diagnostic and public health surveillance, the specific and accurate identification and quantification of proteins in complex biological samples remains a recurrent and challenging problem. For many protein biomarkers, this problem has been solved by immunological techniques. However, the success of immunological approaches relies on the heavy duty production and validation of high specificity and high affinity antibodies. Although recent efforts are being made to design antibodies arrays, the adaptation of immunological methods to multiplexed analyses remains limited. Indeed, the simultaneous optimization of several protein assays is hardly ever possible. Alternatively, the power of mass spectrometry (MS)-based proteomics can be harnessed to determine the presence of specific proteins in a sample.

### BRIEF SUMMARY

Embodiments of the invention relate to methods for identifying and/or quantifying a target polypeptide or target polypeptides in a sample comprising the steps of:
providing a sample to be analyzed;
treating the sample with a protease activity to generate a plurality of proteolytic peptides;
separating the proteolytic peptides via liquid chromatography;
analyzing the separated proteolytic peptides by mass spectrometry (MS); and/or
analyzing the results of the MS to determine the presence of particular target fragments.

In embodiments, a known quantity of an internal standard spike may be added to the sample, thereby generating a spiked sample. In further embodiments, a protein may be identified as being present in the sample where three or more target fragments specific for a particular protein are found among the proteolytic fragments.

These and other aspects of the disclosure will become apparent to the skilled artisan in view of the teachings contained herein.

### DETAILED DESCRIPTION

Embodiments of the invention include methods of detecting the presence of a particular polypeptide in a sample. Examples of proteins that can be detected using the methods described herein include, but are not limited to, α-S1-Casein, β-Lactoglobulin, Vicilin, Glutelin, and Glycinin G1 (SEQ ID NOS: 1-5 of the Sequence Listing incorporated herein, respectively).

The term “target fragment” refers to a specific polypeptide obtained after proteolysis of a polypeptide to be detected, which is a fragment of a larger protein. Examples of target fragments include, but are not limited to, SEQ ID NOS:6-20.

As used herein, a “protease activity” is an activity that cleaves amide bonds in a polypeptide. The activity may be implemented by an enzyme such as a protease or by a chemical agent. Suitable proteases include, but are not limited to one or more of serine proteases (e.g., such as trypsin, hepsin, SCCE, TADG12, TADG14); metalloproteases (e.g., such as PUMP-1); chymotrypsin; cathepsin; pepsin, elastase; pronase; Arg-C; Asp-N; Glu-C; Lys-C; carboxypeptidases A, B, and/or C; dispase; thermolysin; cysteine proteases such as gingipains, and the like. Proteases may be isolated from cells or obtained through recombinant techniques. Chemical agents with a protease activity such as CNBr can also be used. In embodiments, the sample may be subjected to the protease activity until essentially all cleavage sites have been acted upon.

The method described herein may be used in a large variety of fields; such as proteomics, detection of biomarkers in biological samples, quality controls in the manufacture of vaccines and other bioproducts, biological and health hazard controls, food, detection of specific ingredients in foods and/or raw materials, and/or water controls.

Typically, the protein to be detected may be a biomarker, a protein or a fragment thereof which is physically, physiologically, or pathologically present in a sample, a bacterial protein, a viral protein, a plant protein, a yeast protein, a mold protein, a fungal protein, an animal protein or a toxin.

The size of the target fragment may be any size as long as the presence of the target fragment is detectable by the methods described herein. For example, target fragments, may be about 10, 15, 20, 25, 30, 35, 40, or 50 polypeptides in length.

Examples of samples on which the methods may be performed are foods, food ingredients, nutraceuticals, biological fluids (for example, but not limited to, blood, serum, plasma, cerebrospinal fluid, urine, saliva, and lachrymal fluid), tissue and cells homogenates, cell culture supernantants, water, biocollection fluids and any biochemical fraction derived from the above materials. Biocollection fluids are fluids which are used for collecting particles which may be present in air or gas samples.

Examples of foods and food ingredients include, but are not limited to, cow's milk, pea, rice, soy, and wheat.

The method described herein may also allow the simultaneous detection of more than one target fragment. In specific embodiments, the three or more different target fragments may be used in combination to detect the presence of a particular polypeptide in the sample. Multiplex detection of target fragments may also be performed including the detection of one or more proteins via one or more sets of target fragments.

In certain embodiments, a known quantity of standard may be added to the proteolytic fragments before analysis. For example, a known quantity of β-Casomorphin 1-4 may be added to the proteolytic fragments as an internal control. Examples of known quantities of a standard include, but are not limited to about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 350, 400, 450, 500, 1000, 1500, 2000, 2500, 3000, 3500, and 4000 ng/mL of the standard.

In embodiments, the proteolytic peptides may be separated by chromatography prior to analysis with mass spectrometry. Examples of chromatography include, but are not limited to, liquid, affinity, ion exchange, size exclusion, expanded bed adsorption, reversed phase, two-dimensional, simulated moving-bed, fast protein liquid, countercurrent, and chiral chromatography. In certain embodiments, the chromatography may be high-performance liquid chromatography.

Examples of stationary phases used in liquid chromatography include, but are not limited to, alkyl, polar, amide, phenyl, chiral, and ion pairing phases.

In further embodiments, the presence of a target peptide among the proteolytic fragments may be detected by mass spectrometry. Generally, mass spectrometry ionizes chemical species and sorts the ions based on their mass to charge ratio. In this way, specific chemical species (e.g. target fragments) may be detected in a complex sample.

The disclosed methods will become further apparent to the skilled artisan in view of the following examples.

### EXAMPLES

### Example 1: Peptide Selection

To qualitatively identify a protein source, unique peptides (relative to other peptides from other proteins in the assay) that act as markers for specific proteins originating from the source species were chosen. To identify these unique marker peptides, signature protein(s) from each source were identified via literature search. The protein in cow's milk is approximately 80% casein protein and 20% whey protein [1]. These two fractions can be purified separately yielding distinct raw materials. Therefore a signature protein for both the casein and whey fractions was needed. The proteins chosen were α-S1-Casein (Cow Milk, casein fraction) [1,2], β-Lactoglobulin (Cow Milk, whey fraction) [1,2], Vicilin (Pea) [3,4], Glutelin (Rice) [3,5] and Glycinin G1 (Soy) [3,6].

An in silico digestion was performed on the signature proteins to generate tryptic peptides [7]. As high level plant proteins tend to belong to related seed storage protein families they are likely to have homologous amino acid sequences. A Basic Local Alignment Search Tool (BLAST) [8] search was performed to establish peptide specificity at this stage of development. The specific peptides were then analyzed by LC/MS/MS.

### Example 2: Sample Preparation and Testing

Sample Preparation

Samples were thoroughly mixed before weighing. 500 mg of sample was transferred to a 15 mL centrifuge tube. 10 mL of the extraction buffer (300.3 g of Urea (MW=60.06) and 6.057 g Tris base (MW=121.14) were transferred to a 1 liter graduated cylinder and combined with ultrapure water to form 1 Liter of solution) was added and the resulting solution vortexed for 10-60 seconds until the sample material was a homogenous slurry. The slurry was then sonicated for 60 minutes.

After sonication the tubes were spun down for 10 minutes at 4000 g to pellet remaining solids. 6 mL of supernatant was transferred by pipette to a new 15 mL conical centrifuge tube and 200 μL of the thawed reducing solution (1.697 g of dithiotreitol (DTT, MW=154.253) in 11 mL ultrapure water) was added. The resulting solution was vortexed to mix and heated for 30 minutes in a 55° C. water bath. After cooling to room temperature, 2.0 mL of the freshly prepared alkylating solution (185 mg of iodoacetamide (MW=184.96) in 2 mL of ultrapure water) was added to the samples. The samples were then mixed by vortexing and stored with the tube protected from light for 30 minutes at room temperature.

Following alkylation, 6 mL of digestion buffer (3.953 g of ammonium bicarbonate (MW=79.06) in 400 mL ultrapure water) was added to each sample. After carefully mixing by inverting tube multiple times. 400 μL of the thawed trypsin working solution (500 μg/mL in digestion buffer) (200 μg trypsin per sample) was added and mixed by carefully inverting tube multiple times. The samples are then incubated overnight in an incubator at 37° C. with times at start and finish of incubation recorded.

10 mL of each sample were transferred by pipette to a new 15 mL conical centrifuge tube and 417 μL formic acid added and mixed by inverting the tube multiple times to quench the samples. The samples were then centrifuged for 10 minutes at 4000 g to pellet any precipitate. The resulting supernatant was then filtered using a disposable syringe and a 0.22 or 0.45 μm nylon syringe filter into a new 15 mL conical centrifuge tube.

The sample was then diluted 1:10 with 0.1% formic acid and internal standard spike solution (2000 ng/mL B-Casomorphin 1-4 (YPFP) in PBS). Example: Add 100 μL of sample to autosampler vial, add 100 μL of internal standard spike solution and dilute with 800 μL 0.1% formic acid.

Measurement of Target Fragments

Approximately 5 μL of sample was injected into HPLC column linked to mass spectrometer for analysis.

Liquid Chromatoraphic Conditions (Shimadzu Prominence High-performance Liquid Chromatography (HPLC) System)

Column (Phenomenex HPLC column (C18, 150×2 mm, Synergi 4μ Hydro 80 Å, Part Number 00F-4375-B0)) Parameters (Table 1)

| HPLC Column | Luna 3 μm, 150 × 2 mm C18, 200 Å | |
|---|---|---|
| Column Temperature | 30° | C. |
| Recommended Injection Volume | 5 | μL |
| Flow Rate | 0.3 | mL/min |

Pump Gradient (Table 2)

| Time (min) | A (%) | B (%) | Curve |
|---|---|---|---|
| 0.0 | 85 | 15 | 0 |
| 0.8 | 85 | 15 | 0 |
| 5.1 | 50 | 50 | 0 |
| 6.4 | 2 | 98 | 0 |
| 7.3 | 2 | 98 | 0 |
| 7.5 | 85 | 15 | 0 |
| 10 | 85 | 15 | 0 |

Valco Valve 2-Position Schedule

Position A: Flow from column diverted to waste

Position B: Flow from column sent to MS for analysis

Schedule (Table 3)

| Time (min) | Position (A/B) | Destination |
|---|---|---|
| 0 | A | Waste |
| 1 | B | MS analysis |
| 9 | A | Waste |

Mass Spectrometer Conditions (SCIEX 4000 Q TRAP Triple Quadrupole Mass Spectrometer Detector)

Typical Instrument Settings (Table 4)

Fixed

| | | |
|---|---|---|
| Ion Transfer Voltage | IS | 5500 |
| Probe Temperature | TEM | 500 |
| Interface Heater | ihe | ON |

Typical

| | | |
|---|---|---|
| Curtain Gas | CUR | 20 |
| Ion Source Gas 1 | GS1 | 40 |
| Ion Source Gas 1 | GS2 | 50 |
| Collision Gas | CAD | High |

### Example 3: Method Reporting Limit (MRL) Testing

The MRL for each marker peptide was examined in protein free matrices spiked with relevant protein raw materials at 100, 500 and 1000 ppm. These composite spikes were then analyzed to assess the MRL for each marker peptide. The criteria for MRL acceptance was that all three peptides must be present at least one spike level and that the highest blank peak area cannot exceed 20% of the MRL peak area.

A composite sample of 12 separate protein samples was prepared as outlined in Table 5. The protein percent, as determined by Kjeldahl, for each individual protein sample was used to ensure that each protein source was at the same level in the final composite. This protein mix sample was used for precision testing and the creation of spiked samples used for MRL evaluation.

TABLE 5
Composite protein mix of samples used for MRL testing.

| | | Protein % | Weight | Protein | Combined |
|---|---|---|---|---|---|
| Source | SID | (Kjeldahl) | (g) | % | % |
| Cow Milk | 405984 | 80.37% | 17.416 | 7.03% | 21.1% |
| | 410058 | 80.95% | 17.279 | 7.03% | |
| | 410057 | 79.89% | 17.511 | 7.03% | |
| Pea | 409386 | 85.85% | 16.296 | 7.03% | 21.1% |
| | 404987 | 79.25% | 17.661 | 7.03% | |
| | 407133 | 80.17% | 17.452 | 7.03% | |
| Rice | 408894 | 83.71% | 16.729 | 7.04% | 21.1% |
| | 409338 | 85.25% | 16.421 | 7.03% | |
| | 405915 | 85.84% | 16.306 | 7.03% | |
| Soy | 405663 | 92.10% | 15.204 | 7.04% | 21.1% |
| | 409547 | 90.93% | 15.403 | 7.04% | |
| | 395810 | 91.18% | 15.361 | 7.04% | |
| Total | | | 199.039 | 84.39% | |

For MRL evaluation and selectivity testing negative control matrices, either a protein free raw material (pure BCAAs, Branched chain amino acids) or protein free finished good matrix (mix of BCAA raw and a finished good), were used. To evaluate the MRL, the composite protein mix was spiked into negative control matrices as detailed in Table 6. The unspiked negative control matrices were used for selectivity testing

TABLE 6
Spiked protein free negative control matrices used for MRL testing

| | Negative Control | | | | | |
|---|---|---|---|---|---|---|
| | Matrix | | Spike | | Concentration | |
| Name | Material | (g) | Material | (g) | % Protein | ppm |
| RM_1_20000 | BCAA, | 8.964 | Protein Mix | 0.9364 | 1.995% | 19954 |
| RM_1_1000 | 410640 | 9.538 | RM_1_20000 | 0.5016 | 0.100% | 997 |
| RM_1_500 | | 9.731 | RM_1_20000 | 0.2506 | 0.050% | 501 |
| RM_1_100 | | 9.066 | RM_1_1000 | 1.006 | 0.010% | 100 |
| RM_2_20000 | BCAA, | 9.024 | Protein Mix | 0.9473 | 2.004% | 20043 |
| RM_2_1000 | 410641 | 9.493 | RM_2_20000 | 0.5006 | 0.100% | 1004 |
| RM_2_500 | | 9.785 | RM_2_20000 | 0.2490 | 0.050% | 497 |
| RM_2_100 | | 9.002 | RM_2_1000 | 1.0010 | 0.010% | 100 |
| FG_1_20000 | BCAA, | 9.049 | Protein Mix | 0.9470 | 1.999% | 19987 |
| FG_1_1000 | 410642 | 9.520 | FG_1_20000 | 0.5012 | 0.100% | 1000 |
| FG_1_500 | (20.132 g) | 9.754 | FG_1_20000 | 0.2511 | 0.050% | 502 |
| FG_1_100 | FG, | 9.014 | FG_1_1000 | 0.9985 | 0.010% | 100 |
| | 409016 | | | | | |
| | 20.124 g) | | | | | |
| FG_2_20000 | BCAA, | 9.015 | Protein Mix | 0.9457 | 2.003% | 20030 |
| FG_2_1000 | 410642 | 9.541 | FG_2_20000 | 0.5016 | 0.100% | 1000 |
| FG_2_500 | (20.150 g) | 9.765 | FG_2_20000 | 0.2507 | 0.050% | 501 |
| FG_2_100 | FG, | 9.020 | FG_2_1000 | 0.9997 | 0.010% | 100 |
| | 409101 | | | | | |
| | (20.131 g) | | | | | |

The MRL for each marker peptide was examined in raw material (RM) and finished good (FG) negative control matrices, spiked with protein at 100, 500 and 1000 ppm (see Table 6). During MRL testing, a solution blank was injected after each sample. The peak area of marker peptides in blank injections was tracked and the highest blank peak area was used during the MRL assessment. The criteria for acceptance for each protein source were that all three peptides must be present and that the highest blank peak area cannot exceed 20% of the lowest qualifying MRL peak area. The results for MRL testing are shown in Table 7.

TABLE 7
Observed ppm MRLs for marker peptides. Spiked negative
control matrices were tested and the lowest concentration
spike levels for each marker peptide which met the criteria for
acceptance is shown. Samples are described in Table 6.

| | Cow Milk - | | | Cow Milk - | | | | | | | | | | | |
|---|---|---|---|---|---|---|---|---|---|---|---|---|---|---|---|
| | Casein | | | Whey | | | Rice | | | Pea | | | Soy | | |
| Sample | Pep1 | Pep2 | Pep3 | Pep1 | Pep2 | Pep3 | Pep1 | Pep2 | Pep3 | Pep1 | Pep2 | Pep3 | Pep1 | Pep2 | Pep3 |
| RM-1 | 1000 | 1000 | 500 | 1000 | 500 | 500 | 500 | 100 | 500 | 100 | 100 | 100 | 500 | 100 | 100 |
| RM-2 | 1000 | 1000 | 500 | 500 | 500 | 500 | 100 | 100 | ND | 100 | 100 | 100 | 1000 | 100 | 500 |
| FG-1 | 1000 | ND | 500 | 500 | 500 | 500 | 100 | 100 | 500 | 100 | 100 | 100 | 1000 | 100 | 100 |
| FG-2 | 1000 | 1000 | 500 | 500 | 500 | 500 | 100 | 100 | 500 | 100 | 100 | 500 | 1000 | 100 | 100 |

These results indicate that the marker peptides exhibit different MRLs. In all protein free matrices, more than one peptide was assigned to each of the ppm levels tested. While further testing might identify an exact MRL, for our purposes this semiquantitative approach is sufficient as it is a conservative measure of where the lowest concentration limit can be detected. For testing, on a per assay basis the MRL will be examined using the three spike levels in the appropriate protein free matrix. This necessitates the creation of laboratory control samples (LCS) that can be used on an ongoing basis. From this testing, RM-1 and FG-2 appear appropriate for use as LCS as MRLs can be assigned for each of the marker peptides. For samples in which MRL could not be assigned for all marker peptides (Cow Milk-Casein Peptide 2 in FG-1 and Rice Peptide 3 in RM-2) these samples are deemed inappropriate for use as LCS. A complete tabulation of the MRL results including blanks are contained within Table 8.

TABLE 8
MRL Testing, Tabulated Data

| | Cow Milk-Casein | | | Cow Milk-Whey | | |
|---|---|---|---|---|---|---|
| Sample Name | Pep1 | Pep2 | Pep3 | Pep1 | Pep2 | Pep3 |
| DMSO Blank | 45800 | 29500 | 5270 | 455 | 1040 | 219 |
| 0.1% FA Blank | 9340 | 3910 | 666 | 33 | 0 | 211 |
| BCAA_1_400640 | 10300 | 5040 | 812 | 0 | 0 | 195 |
| 0.1% FA Blank | 9970 | 4750 | 552 | 0 | 146 | 127 |
| BCAA_2_400641 | 11900 | 5290 | 1100 | 33 | 341 | 244 |
| 0.1% FA Blank | 8690 | 5690 | 449 | 33 | 244 | 130 |
| BCAA_400642_C4_409016 | 11700 | 3610 | 717 | 0 | 211 | 0 |
| 0.1% FA Blank | 11100 | 5650 | 725 | 0 | 130 | 114 |
| BCAA_400642_C4_409101 | 11200 | 4430 | 541 | 0 | 195 | 244 |
| 0.1% FA Blank | 10800 | 5790 | 552 | 33 | 179 | 141 |
| DMSO Blank | 14100 | 12700 | 1860 | 308 | 179 | 276 |
| 0.1% FA Blank | 11100 | 5000 | 698 | 0 | 114 | 195 |
| RM_1_100 | 19300 | 12900 | 2090 | 195 | 357 | 520 |
| 0.1% FA Blank | 11600 | 6230 | 612 | 65 | 179 | 195 |
| RM_2_100 | 18200 | 12300 | 1610 | 130 | 211 | 695 |
| 0.1% FA Blank | 13300 | 6000 | 831 | 33 | 114 | 260 |
| FG_1_100 | 20700 | 12000 | 1610 | 49 | 438 | 641 |
| 0.1% FA Blank | 12800 | 4770 | 649 | 65 | 244 | 276 |
| FG_2_100 | 20100 | 8370 | 1540 | 179 | 419 | 335 |
| 0.1% FA Blank | 15400 | 6260 | 641 | 65 | 244 | 276 |
| DMSO Blank | 17900 | 12000 | 1740 | 325 | 308 | 127 |
| 0.1% FA Blank | 13900 | 5360 | 568 | 49 | 162 | 71 |
| RM_1_500 | 69100 | 37800 | 5690 | 341 | 1830 | 2760 |
| 0.1% FA Blank | 13000 | 6660 | 744 | 49 | 192 | 146 |
| RM_2_500 | 63700 | 41000 | 4780 | 649 | 1630 | 2060 |
| 0.1% FA Blank | 10200 | 7820 | 392 | 0 | 81 | 244 |
| FG_1_500 | 54900 | 35500 | 6220 | 503 | 1590 | 1720 |
| 0.1% FA Blank | 13100 | 9650 | 503 | 97 | 81 | 179 |
| FG_2_500 | 80700 | 43200 | 6200 | 552 | 1990 | 2460 |
| 0.1% FA Blank | 14100 | 10100 | 587 | 81 | 97 | 230 |
| DMSO Blank | 15400 | 17100 | 1620 | 243 | 227 | 198 |
| 0.1% FA Blank | 11300 | 8680 | 579 | 81 | 0 | 227 |
| RM_1_1000 | 116000 | 67800 | 10800 | 1100 | 2670 | 3850 |
| 0.1% FA Blank | 12400 | 9760 | 890 | 0 | 195 | 143 |
| RM_2_1000 | 133000 | 77700 | 12500 | 974 | 3190 | 4330 |
| 0.1% FA Blank | 11600 | 7470 | 633 | 33 | 222 | 195 |
| FG_1_1000 | 112000 | 46100 | 11000 | 1280 | 3570 | 4470 |
| 0.1% FA Blank | 13200 | 11900 | 747 | 0 | 130 | 141 |
| FG_2_1000 | 107000 | 68100 | 10200 | 1010 | 2660 | 3340 |
| 0.1% FA Blank | 16500 | 9400 | 679 | 81 | 162 | 114 |
| DMSO Blank | 15600 | 14000 | 1510 | 195 | 179 | 227 |
| 0.1% FA Blank | 12600 | 11900 | 673 | 64.90 | 162 | 130 |

| | Rice | | | Pea | | | Soy | | |
|---|---|---|---|---|---|---|---|---|---|
| Sample Name | Pep1 | Pep2 | Pep3 | Pep1 | Pep2 | Pep3 | Pep1 | Pep2 | Pep3 |
| DMSO Blank | 130 | 0 | 0 | 49 | 227 | 0 | 211 | 192 | 0 |
| 0.1% FA Blank | 0 | 0 | 0 | 0 | 0 | 0 | 0 | 0 | 0 |
| BCAA_1_400640 | 0 | 0 | 0 | 0 | 0 | 0 | 0 | 0 | 0 |
| 0.1% FA Blank | 0 | 0 | 0 | 0 | 0 | 0 | 0 | 0 | 0 |
| BCAA_2_400641 | 0 | 0 | 0 | 0 | 0 | 0 | 0 | 0 | 0 |
| 0.1% FA Blank | 0 | 0 | 0 | 0 | 0 | 0 | 114 | 0 | 0 |
| BCAA_400642_C4_409016 | 0 | 0 | 0 | 33 | 0 | 0 | 0 | 0 | 0 |
| 0.1% FA Blank | 0 | 0 | 0 | 0 | 0 | 0 | 0 | 33 | 0 |
| BCAA_400642_C4_409101 | 0 | 0 | 0 | 0 | 0 | 0 | 0 | 0 | 0 |
| 0.1% FA Blank | 0 | 0 | 0 | 0 | 0 | 0 | 0 | 0 | 0 |
| DMSO Blank | 0 | 0 | 0 | 97 | 0 | 0 | 438 | 114 | 0 |
| 0.1% FA Blank | 0 | 0 | 0 | 0 | 0 | 0 | 0 | 0 | 0 |
| RM_1_100 | 0 | 584 | 0 | 552 | 471 | 114 | 114 | 601 | 81 |
| 0.1% FA Blank | 0 | 0 | 0 | 0 | 0 | 0 | 0 | 33 | 0 |
| RM_2_100 | 146 | 720 | 0 | 536 | 417 | 162 | 97 | 682 | 0 |
| 0.1% FA Blank | 0 | 0 | 0 | 0 | 0 | 0 | 0 | 0 | 0 |
| FG_1_100 | 162 | 882 | 0 | 195 | 487 | 81 | 81 | 390 | 97 |
| 0.1% FA Blank | 0 | 0 | 0 | 0 | 0 | 0 | 0 | 0 | 0 |
| FG_2_100 | 81 | 855 | 0 | 244 | 244 | 0 | 0 | 341 | 65 |
| 0.1% FA Blank | 0 | 0 | 0 | 0 | 0 | 0 | 0 | 33 | 0 |
| DMSO Blank | 0 | 0 | 0 | 97 | 0 | 0 | 257 | 114 | 0 |
| 0.1% FA Blank | 0 | 0 | 0 | 0 | 0 | 0 | 0 | 0 | 0 |
| RM_1_500 | 509 | 4740 | 97 | 2770 | 1850 | 520 | 698 | 4160 | 771 |
| 0.1% FA Blank | 0 | 0 | 0 | 0 | 0 | 0 | 0 | 0 | 0 |
| RM_2_500 | 774 | 3620 | 0 | 2200 | 2790 | 749 | 514 | 2670 | 520 |
| 0.1% FA Blank | 0 | 0 | 0 | 0 | 0 | 0 | 0 | 0 | 0 |
| FG_1_500 | 552 | 3920 | 97 | 796 | 1670 | 487 | 179 | 1300 | 487 |
| 0.1% FA Blank | 0 | 0 | 0 | 0 | 0 | 0 | 0 | 0 | 0 |
| FG_2_500 | 511 | 5540 | 49 | 1610 | 2020 | 503 | 438 | 2730 | 904 |
| 0.1% FA Blank | 0 | 0 | 0 | 0 | 0 | 0 | 0 | 0 | 0 |
| DMSO Blank | 0 | 0 | 0 | 195 | 0 | 0 | 227 | 81 | 0 |
| 0.1% FA Blank | 0 | 0 | 0 | 0 | 0 | 0 | 65 | 33 | 0 |
| RM_1_1000 | 1400 | 9840 | 130 | 4520 | 5390 | 1400 | 1720 | 7040 | 1530 |
| 0.1% FA Blank | 0 | 0 | 0 | 0 | 0 | 0 | 114 | 0 | 0 |
| RM_2_1000 | 1650 | 6280 | 0 | 4830 | 3850 | 1460 | 1040 | 6870 | 1460 |
| 0.1% FA Blank | 0 | 0 | 0 | 0 | 0 | 0 | 0 | 0 | 0 |
| FG_1_1000 | 1050 | 8120 | 81 | 2400 | 3730 | 1410 | 852 | 4570 | 1310 |
| 0.1% FA Blank | 0 | 0 | 0 | 0 | 0 | 0 | 0 | 0 | 0 |
| FG_2_1000 | 863 | 8470 | 130 | 2480 | 3060 | 1380 | 1340 | 6030 | 1130 |
| 0.1% FA Blank | 0 | 0 | 0 | 0 | 0 | 0 | 0 | 0 | 0 |
| DMSO Blank | 0 | 0 | 0 | 127 | 0 | 81 | 179 | 97 | 0 |
| 0.1% FA Blank | 0 | 0 | 0 | 0 | 0 | 0 | 0 | 32.5 | 0 |

### Example 3: Selectivity

The following samples were used to validate selectivity, specificity, precision, and reinjection reproducibility (Table 9).

| Sample | | | Protein % |
|---|---|---|---|
| ID | Species (source) | Sample Description | (Kjeldahl) |
| 405984 | Bos taurus | Milk Protein Conc 80% | 80.37% |
| 410058 | (Cow) | Milk Protein Conc 80% | 80.95% |
| 410057 | | Milk Protein Conc 80% | 79.89% |
| 409386 | Pisum sativum | Pea Protein 80% | 85.85% |
| 404987 | (Pea) | GRN Vegotein 80% | 79.25% |
| 407133 | | GRN Vegotein 80% | 80.17% |
| 408894 | Oryza sativa | Rice Protein 80% | 83.71% |
| 409338 | (Rice) | GRN Organic Oryzatein 90% | 85.25% |
| 405915 | | GRN Organic Oryzatein 90% | 85.84% |
| 405663 | Glycine max | SoyPura 310 | 92.10% |
| 409547 | (Soy) | Supro 661 IP | 90.93% |
| 395810 | | Supro 661 IP | 91.18% |
| 410640 | None. Blend of | iBCAA 2:1:1 | (0%) |
| 410641 | amino acids (LEU, | iBCAA 2:1:1 | (0%) |
| 410642 | ILE, VAL). | iBCAA 2:1:1 | (0%) |
| 409016 | None. | WBT C4 Mass BR | (0%) |
| 409101 | | WBT C4 Mass FP | (0%) |

TABLE 10
Optimized MRM parameters used for marker peptide detection.

| | | | Q1 | Q3 | CE |
|---|---|---|---|---|---|
| Protein (source) | Peptide | Amino Acid Sequence | (m/z) | (m/z) | (V) |
| α-S1-Casein | 1 | FFVAPFPEVFKG | 692.9 | 920.5 | 29 |
| (Cow Milk) | | (SEQ ID NO: 6) | | | |
| | 2 | YLGYLEQLLR | 634.4 | 991.6 | 33 |
| | | (SEQ ID NO: 7) | | | |
| | 3 | HQGLPQEVLNENLLR | 880.5 | 1324.7 | 51 |
| | | (SEQ ID NO: 8) | | | |
| β-Lactoglobulin | 1 | VYVEELKPTPEGDLEILLQK | 1157.1 | 1453 | 60 |
| (Cow Milk) | | (SEQ ID NO: 9) | | | |
| | 2 | VLVLDTDYK | 533.3 | 853.5 | 23 |
| | | (SEQ ID NO: 10) | | | |
| | 3 | LIVTQTMK | 467.3 | 707.4 | 21 |
| | | (SEQ ID NO: 11) | | | |
| Vicilin | 1 | EGSLLLPHYNSR | 693.4 | 773.6 | 40 |
| (Pea) | | (SEQ ID NO: 12) | | | |
| | 2 | GDFELVGQR | 510.8 | 572.5 | 26 |
| | | (SEQ ID NO: 13) | | | |
| | 3 | GPIYSNEFGK | 556.3 | 844.5 | 29 |
| | | (SEQ ID NO: 14) | | | |
| Glutelin | 1 | ALPNDVLANAYR | 658.9 | 566.8 | 26 |
| (Rice) | | (SEQ ID NO: 15) | | | |
| | 2 | LQAFEPIR | 487.3 | 732.5 | 23 |
| | | (SEQ ID NO: 16) | | | |
| | 3 | GDEFGAFTPIQYK | 736.9 | 1024.8 | 33 |
| | | (SEQ ID NO: 17) | | | |
| Glycinin G1 | 1 | VLIVPQNFVVAAR | 713.4 | 1001.6 | 33 |
| (Soy) | | (SEQ ID NO: 19) | | | |
| | 2 | VFDGELQEGR | 575.3 | 903.4 | 29 |
| | | (SEQ ID NO: 19) | | | |
| | 3 | LNALKPDNR | 520.8 | 629.3 | 32 |
| | | (SEQ ID NO: 20) | | | |

The chromatograms for negative control matrices were examined to assess the selectivity of the method. For each negative control matrix, the criteria was that no peaks for the marker peptides at the respective retention time could exceed 20% of the lowest qualifying MRL peak area. During MRL testing, it was determined that for specific peptides in RM-2 and FG-1 MRL values could not be assigned. The MRL peak areas for RM-1 spikes were used to assess the selectivity of RM-1 and RM-2 negative control samples while FG-2 spikes were used for FG-1 and FG-2 negative control samples. The selectivity results are shown in Table 11, with the percentage of the MRL peak area for any relevant peaks. Full tabulated results of selectivity are shown in Tables 12 and 13.

TABLE 11
Summary of selectivity testing of negative control matrices. The results are
expressed as percentage of the lowest qualifying MRL peak area in the
appropriate matrix. For RM-1 and RM-2 negative control samples, the MRL
peak areas in RM-1 were used. For FG-1 and FG-2 negative control samples,
the MRL peak areas in FG-2 were used. For marker peptides that were not
present in the negative control samples a value of 0% was assigned.

| | Cow Milk - | | | Cow Milk - | | | | | | | | | | | |
|---|---|---|---|---|---|---|---|---|---|---|---|---|---|---|---|
| | Casein | | | Whey | | | Rice | | | Pea | | | Soy | | |
| | Pep1 | Pep2 | Pep3 | Pep1 | Pep2 | Pep3 | Pep1 | Pep2 | Pep3 | Pep1 | Pep2 | Pep3 | Pep1 | Pep2 | Pep3 |
| RM-1 | 9% | 7% | 14% | 0% | 0% | 7% | 0% | 0% | 0% | 0% | 0% | 0% | 0% | 0% | 0% |
| RM-2 | 10% | 8% | 19% | 3% | 19% | 9% | 0% | 0% | 0% | 0% | 0% | 0% | 0% | 0% | 0% |
| FG-1 | 11% | 5% | 12% | 0% | 11% | 0% | 0% | 0% | 0% | 13% | 0% | 0% | 0% | 0% | 0% |
| FG-2 | 10% | 7% | 9% | 0% | 10% | 10% | 0% | 0% | 0% | 0% | 0% | 0% | 0% | 0% | 0% |

No peaks were observed in the negative control samples whose peak area was greater than 20% of the lowest qualifying MRL peak area. The method was therefore determined to be selective and free from reagent impurities or matrix effects.

TABLE 12
MRL Peak areas for spikes into negative control matrix

| | Cow Milk - | | | Cow Milk- | | | | | | | | | | | |
|---|---|---|---|---|---|---|---|---|---|---|---|---|---|---|---|
| | Casein | | | Whey | | | Rice | | | Pea | | | Soy | | |
| | Pep1 | Pep2 | Pep3 | Pep1 | Pep2 | Pep3 | Pep1 | Pep2 | Pep3 | Pep1 | Pep2 | Pep3 | Pep1 | Pep2 | Pep3 |
| RM-1 | 116000 | 67800 | 5690 | 1100 | 1830 | 2760 | 509 | 584 | 97 | 552 | 471 | 114 | 698 | 601 | 81 |
| FG-2 | 107000 | 68100 | 6200 | 552 | 1990 | 2460 | 81 | 855 | 49 | 244 | 244 | 503 | 1340 | 341 | 65 |

TABLE 13
Peak areas for peaks detected in negative control matrix samples

| | Cow Milk - Casein | | | Cow Milk - Whey | | | Rice | | | Pea | | | Soy | | |
|---|---|---|---|---|---|---|---|---|---|---|---|---|---|---|---|
| | Pep1 | Pep2 | Pep3 | Pep1 | Pep2 | Pep3 | Pep1 | Pep2 | Pep3 | Pep1 | Pep2 | Pep3 | Pep1 | Pep2 | Pep3 |
| RM-1 | 10300 | 5040 | 812 | 0 | 0 | 195 | 0 | 0 | 0 | 0 | 0 | 0 | 0 | 0 | 0 |
| RM-2 | 11900 | 5290 | 1100 | 33 | 341 | 244 | 0 | 0 | 0 | 0 | 0 | 0 | 0 | 0 | 0 |
| FG-1 | 11700 | 3610 | 717 | 0 | 211 | 0 | 0 | 0 | 0 | 33 | 0 | 0 | 0 | 0 | 0 |
| FG-2 | 11200 | 4430 | 541 | 0 | 195 | 244 | 0 | 0 | 0 | 0 | 0 | 0 | 0 | 0 | 0 |

### Example 4: Specificity

The specificity of the qualitative method was established through examination of individual raw material samples from various protein sources. The criteria for specificity was that for each raw material sample, peaks for all three source marker peptides must be present with peak area greater than the MRL peak area, and that for other marker peptides no peaks with area greater than the MRL should be present. For each raw material sample, the signal was compared to the MRL peak areas in RM-1 spikes. Specificity results are shown in Table 14. Full specificity data sets are contained within Tables 15 and 16.

TABLE 14
Specificity testing. Shown are the results for three lots of protein powder
from each of the four source species. A “+” result was assigned
to marker peptides whose peak areas was greater than the MRL.

| | | Cow Milk - | | | Cow Milk - | | | | | | | | | | | |
|---|---|---|---|---|---|---|---|---|---|---|---|---|---|---|---|---|
| | | Casein | | | Whey | | | Rice | | | Pea | | | Soy | | |
| | SID | Pep1 | Pep2 | Pep3 | Pep1 | Pep2 | Pep3 | Pep1 | Pep2 | Pep3 | Pep1 | Pep2 | Pep3 | Pep1 | Pep2 | Pep3 |
| Milk | 405984 | + | + | + | + | + | + | | | | + | | | | | |
| | 410058 | + | + | + | + | + | + | | | | + | | | | | |
| | 410057 | + | + | + | + | + | + | | | | | | | | | |
| Rice | 408894 | | | | | | | + | + | + | | | | | | |
| | 409338 | | | | | | | + | + | + | | | | | | |
| | 405915 | | | | | | | + | + | + | | | + | | | |
| Pea | 409386 | | | | | + | | + | | | + | + | + | + | + | + |
| | 404987 | | | | | + | | + | | | + | + | + | + | + | |
| | 407133 | | | | | + | | + | | | + | + | + | | | |
| Soy | 405663 | | | | | + | | | | | | | | + | + | + |
| | 409547 | | | | | + | | | | | | | + | + | + | + |
| | 395810 | | | | | + | | | | | | + | + | + | + | + |

TABLE 15
Specificity Testing (peak area) included are MRL peak areas for RM-1

| | Cow Milk - Casein | | | Cow Milk - Whey | | | Rice | | |
|---|---|---|---|---|---|---|---|---|---|
| Sample | Pep1 | Pep2 | Pep3 | Pep1 | Pep2 | Pep3 | Pep1 | Pep2 | Pep3 |
| BCAA:400640 | 116000 | 67800 | 5690 | 1100 | 1830 | 2760 | 509 | 584 | 97 |
| 405984_Milk | 20400000 | 15500000 | 5550000 | 752000 | 1830000 | 1630000 | 0 | 0 | 0 |
| 410058_Milk | 21500000 | 15600000 | 5640000 | 762000 | 1850000 | 1810000 | 0 | 0 | 0 |
| 410057_Milk | 21400000 | 15700000 | 5740000 | 792000 | 2040000 | 1700000 | 0 | 0 | 0 |
| 408894_Rice | 8500 | 5310 | 1050 | 0 | 273 | 476 | 815000 | 3050000 | 86200 |
| 409338_Rice | 8240 | 5570 | 652 | 0 | 365 | 1000 | 1020000 | 6310000 | 113000 |
| 405915_Rice | 9480 | 4710 | 682 | 130 | 476 | 963 | 893000 | 6340000 | 107000 |
| 409386_Pea | 10300 | 6830 | 1040 | 179 | 3110 | 476 | 1180 | 0 | 0 |
| 404987_Pea | 8860 | 6140 | 931 | 195 | 3470 | 446 | 2220 | 0 | 0 |
| 407133_Pea | 9160 | 5680 | 766 | 0 | 3600 | 379 | 1170 | 195 | 0 |
| 405663_Soy | 14600 | 6910 | 1330 | 260 | 2800 | 222 | 352 | 576 | 0 |
| 409547_Soy | 9070 | 5070 | 1230 | 244 | 1990 | 769 | 406 | 303 | 0 |
| 395810_Soy | 9620 | 4600 | 678 | 0 | 2700 | 555 | 0 | 401 | 0 |

| | Pea | | | Soy | | |
|---|---|---|---|---|---|---|
| Sample | Pep1 | Pep2 | Pep3 | Pep1 | Pep2 | Pep3 |
| BCAA:400640 | 552 | 471 | 114 | 698 | 601 | 81 |
| 405984_Milk | 782 | 0 | 0 | 0 | 0 | 0 |
| 410058_Milk | 601 | 0 | 0 | 0 | 0 | 0 |
| 410057_Milk | 471 | 0 | 0 | 0 | 0 | 0 |
| 408894_Rice | 0 | 309 | 0 | 0 | 0 | 0 |
| 409338_Rice | 357 | 0 | 0 | 357 | 0 | 0 |
| 405915_Rice | 390 | 0 | 571 | 0 | 0 | 0 |
| 409386_Pea | 1980000 | 2460000 | 559000 | 4220 | 1960 | 555 |
| 404987_Pea | 2040000 | 2290000 | 527000 | 1420 | 731 | 0 |
| 407133_Pea | 2170000 | 2390000 | 586000 | 0 | 0 | 0 |
| 405663_Soy | 0 | 441 | 0 | 5380000 | 2400000 | 656000 |
| 409547_Soy | 0 | 0 | 384 | 8410000 | 3120000 | 969000 |
| 395810_Soy | 0 | 1090 | 238 | 8610000 | 3270000 | 954000 |

TABLE 16
Specificity Results Expressed as Ratio of MRL peak area

| | Cow Milk - Casein | | | Cow Milk - Whey | | | Rice | | | Pea | | | Soy | | |
|---|---|---|---|---|---|---|---|---|---|---|---|---|---|---|---|
| Sample | Pep1 | Pep2 | Pep3 | Pep1 | Pep2 | Pep3 | Pep1 | Pep2 | Pep3 | Pep1 | Pep2 | Pep3 | Pep1 | Pep2 | Pep3 |
| 405984_Milk | 176 | 229 | 975 | 684 | 1000 | 591 | 0 | 0 | 0 | 1 | 0 | 0 | 0 | 0 | 0 |
| 410058_Milk | 185 | 230 | 991 | 693 | 1011 | 656 | 0 | 0 | 0 | 1 | 0 | 0 | 0 | 0 | 0 |
| 410057_Milk | 184 | 232 | 1009 | 720 | 1115 | 616 | 0 | 0 | 0 | 1 | 0 | 0 | 0 | 0 | 0 |
| 408894_Rice | 0 | 0 | 0 | 0 | 0 | 0 | 1601 | 5223 | 885 | 0 | 1 | 0 | 0 | 0 | 0 |
| 409338_Rice | 0 | 0 | 0 | 0 | 0 | 0 | 2004 | 10805 | 1160 | 1 | 0 | 0 | 1 | 0 | 0 |
| 405915_Rice | 0 | 0 | 0 | 0 | 0 | 0 | 1754 | 10856 | 1099 | 1 | 0 | 5 | 0 | 0 | 0 |
| 409386_Pea | 0 | 0 | 0 | 0 | 2 | 0 | 2 | 0 | 0 | 3587 | 5223 | 4904 | 6 | 3 | 7 |
| 404987_Pea | 0 | 0 | 0 | 0 | 2 | 0 | 4 | 0 | 0 | 3696 | 4862 | 4623 | 2 | 1 | 0 |
| 407133_Pea | 0 | 0 | 0 | 0 | 2 | 0 | 2 | 0 | 0 | 3931 | 5074 | 5140 | 0 | 0 | 0 |
| 405663_Soy | 0 | 0 | 0 | 0 | 2 | 0 | 1 | 1 | 0 | 0 | 1 | 0 | 7708 | 3993 | 8079 |
| 409547_Soy | 0 | 0 | 0 | 0 | 1 | 0 | 1 | 1 | 0 | 0 | 0 | 3 | 12049 | 5191 | 11933 |
| 395810_Soy | 0 | 0 | 0 | 0 | 1 | 0 | 0 | 1 | 0 | 0 | 2 | 2 | 12335 | 5441 | 11749 |

For 3 out of the 12 samples, no marker peptides other than the source peptides were seen above the respective MRLs. For these samples, the method behaved as expected. For 8 out of the 12 samples tested, one or two peptides from a set of non-source marker peptides were seen above the MRL. While the method did not pass the specificity criteria for these samples, identification relies upon peaks at greater than MRL level for all three of the specific marker peptides for that protein source. In those cases where only one (or even two) of the three maker peptide for a specified source is seen, the lack of signal for the other marker peptide(s) indicates that the specific protein is unlikely present in the sample. Thus, specificity of identification is maintained even while the single marker peptide specificity did not perform as expected.

In one case (SID 409386, pea) all three peptides were observed for both pea and soy above the MRL. In this pea sample, the signal for all three pea peptides was >3,500 times the MRL, while for the soy peptides the signal was only 3-7 times the MRL. These soy levels suggest the possibility that the sample contained low level contaminants. The explanation for this observation is not that there is not method specificity, but in fact may be due to the sample itself. This sample is not a true standard which further supports this conclusion, but does not confirm it.

### Example 5: Precision

The precision of the qualitative method was evaluated by examining the % RSD for marker peptide peak areas for intraday analysis of both high and low protein level samples. On one day, six replicate preparations of the initial protein mix (high) and the 1000 ppm spiked composite RM-1 (low) were taken through digestion. The next day these samples were analyzed in parallel. The summarized results are shown in Table 17. Full precision data sets are contained within Table 18.

TABLE 17
Precision testing. Shown is the relative standard deviation from intraday
testing of high (pro mix) and low (RM-1, 1000) protein samples

| Sample | Cow Milk-Casein | | | Cow Milk-Whey | | | Rice | | | Pea | | | Soy | | |
|---|---|---|---|---|---|---|---|---|---|---|---|---|---|---|---|
| Name | Pep1 | Pep2 | Pep3 | Pep1 | Pep2 | Pep3 | Pep1 | Pep2 | Pep3 | Pep1 | Pep2 | Pep3 | Pep1 | Pep2 | Pep3 |
| Low | 9% | 15% | 15% | 29% | 12% | 16% | 28% | 19% | 179% | 15% | 9% | 3% | 22% | 19% | 66% |
| RM-1, 1000 | | | | | | | | | | | | | | | |
| % RSD | | | | | | | | | | | | | | | |
| High | 4% | 5% | 5% | 6% | 8% | 10% | 4% | 5% | 6% | 5% | 7% | 7% | 5% | 5% | 6% |
| PRO MIX | | | | | | | | | | | | | | | |
| % RSD | | | | | | | | | | | | | | | |

TABLE 18
Precision Testing, Tabulated Data of Peak Areas

| | Milk-Casein | | | Milk-Whey | | | Rice | | |
|---|---|---|---|---|---|---|---|---|---|
| Sample Name | Pep1 | Pep2 | Pep3 | Pep1 | Pep2 | Pep3 | Pep1 | Pep2 | Pep3 |
| RM-1, 1000, #1 | 118000 | 80600 | 10200 | 909 | 3280 | 5390 | 790 | 996 | 0 |
| RM-1, 1000, #2 | 118000 | 56000 | 10200 | 714 | 2810 | 4680 | 336 | 1210 | 0 |
| RM-1, 1000, #3 | 96600 | 54400 | 6840 | 1080 | 3390 | 4900 | 525 | 747 | 0 |
| RM-1, 1000, #4 | 101000 | 61300 | 9090 | 587 | 2710 | 3700 | 527 | 823 | 179 |
| RM-1, 1000, #5 | 109000 | 66300 | 10800 | 1340 | 2790 | 3600 | 471 | 763 | 0 |
| RM-1, 1000, #6 | 121000 | 63400 | 10200 | 860 | 3600 | 4490 | 598 | 990 | 65 |
| AVG | 110600 | 63667 | 9555 | 915 | 3097 | 4460 | 541 | 922 | 41 |
| SD | 10084 | 9423 | 1441 | 268 | 374 | 696 | 150 | 178 | 73 |
| RSD | 9% | 15% | 15% | 29% | 12% | 16% | 28% | 19% | 179% |
| PRO MIX, #1 | 13900000 | 8300000 | 2120000 | 279000 | 592000 | 803000 | 143000 | 211000 | 13800 |
| PRO MIX, #2 | 15400000 | 9340000 | 2360000 | 317000 | 718000 | 990000 | 155000 | 244000 | 15700 |
| PRO MIX, #3 | 14900000 | 9130000 | 2260000 | 318000 | 709000 | 1010000 | 148000 | 238000 | 14400 |
| PRO MIX, #4 | 15200000 | 9020000 | 2410000 | 303000 | 732000 | 1000000 | 158000 | 247000 | 15400 |
| PRO MIX, #5 | 15700000 | 9560000 | 2460000 | 332000 | 763000 | 1100000 | 159000 | 237000 | 13900 |
| PRO MIX, #6 | 14700000 | 8920000 | 2370000 | 308000 | 707000 | 995000 | 154000 | 240000 | 15700 |
| AVG | 14966667 | 9045000 | 2330000 | 309500 | 703500 | 983000 | 152833 | 236167 | 14817 |
| SD | 631401 | 431451 | 122311 | 17942 | 58374 | 97263 | 6178 | 12891 | 889 |
| RSD | 4% | 5% | 5% | 6% | 8% | 10% | 4% | 5% | 6% |

| | Pea | | | Soy | | |
|---|---|---|---|---|---|---|
| Sample Name | Pep1 | Pep2 | Pep3 | Pep1 | Pep2 | Pep3 |
| RM-1, 1000, #1 | 3410 | 4240 | 1090 | 1340 | 4830 | 49 |
| RM-1, 1000, #2 | 4630 | 4560 | 1190 | 817 | 4180 | 1090 |
| RM-1, 1000, #3 | 3260 | 3900 | 1130 | 1190 | 4030 | 585 |
| RM-1, 1000, #4 | 3850 | 4190 | 1140 | 1060 | 3730 | 771 |
| RM-1, 1000, #5 | 4470 | 4590 | 1110 | 1590 | 3690 | 254 |
| RM-1, 1000, #6 | 3450 | 5010 | 1140 | 1330 | 5930 | 616 |
| AVG | 3845 | 4415 | 1133 | 1221 | 4398 | 561 |
| SD | 582 | 388 | 34 | 265 | 856 | 370 |
| RSD | 15% | 9% | 3% | 22% | 19% | 66% |
| PRO MIX, #1 | 688000 | 808000 | 194000 | 2410000 | 980000 | 227000 |
| PRO MIX, #2 | 787000 | 919000 | 214000 | 2820000 | 1110000 | 252000 |
| PRO MIX, #3 | 771000 | 920000 | 225000 | 2720000 | 1070000 | 255000 |
| PRO MIX, #4 | 764000 | 951000 | 228000 | 2640000 | 1080000 | 253000 |
| PRO MIX, #5 | 780000 | 986000 | 236000 | 2660000 | 1130000 | 264000 |
| PRO MIX, #6 | 775000 | 980000 | 225000 | 2650000 | 1090000 | 271000 |
| AVG | 760833 | 927333 | 220333 | 2650000 | 1076667 | 253667 |
| SD | 36526 | 65022 | 14706 | 135351 | 52026 | 14989 |
| RSD | 5% | 7% | 7% | 5% | 5% | 6% |

For the composite sample spiked at 1000 ppm, the majority of marker peptides, 10 of 15, showed acceptable precision % RSD≦20%. The % RSD value represents the combined variability of the sample preparation, instrument performance and sample homogeneity. These results suggest that precision is only as reliable as the results in the table for the 5 peptides that exceeded acceptable precision.

Typically samples are expected to contain 20-80% protein, so the protein mix sample is appropriate for examining the precision of the method for regular analysis. All marker peptides in the protein mix sample had % RSD≦10%. This indicates that at the higher protein levels (˜20% protein), the qualitative identification method performed with acceptable precision.

### Example 6: Reinjection Reproducibility

To assess reinjection reproducibility, the primary extracts for the raw material samples that had been stored in the refrigerator at 4° C. for five days were taken through the final dilution step and analyzed. The results for the stored sample were to be deemed acceptable if peaks for all three marker peptides were present with peak areas greater than the MRL.

In each reinjected sample, the appropriate marker peptides had peak areas greater than the MRL. The peak areas were compared to the original data and showed relative responses of approximately 70-130%. These results indicate that for qualitative identification the extracts can be reexamined up to 5 days later if stored appropriately. The reinjection results are shown in Table 19. Full reinjection data sets are contained within Tables 20 and 21.

TABLE 19
Reinjection reproducibility testing. Shown are the results for the source marker peptide
for the reinjection of raw material samples. Results are expressed as a percentage
of the original marker peptide response.

| | | Cow Milk-Casein | | | Cow Milk-Whey | | | Rice | | | Pea | | | Soy | | |
|---|---|---|---|---|---|---|---|---|---|---|---|---|---|---|---|---|
| | SID | Pep1 | Pep2 | Pep3 | Pep1 | Pep2 | Pep3 | Pep1 | Pep2 | Pep3 | Pep1 | Pep2 | Pep3 | Pep1 | Pep2 | Pep3 |
| Milk | 405984 | 108 | 109 | 116 | 127 | 119 | 72 | | | | | | | | | |
| | 410057 | 107 | 110 | 117 | 122 | 124 | 72 | | | | | | | | | |
| | 410058 | 104 | 106 | 113 | 120 | 112 | 78 | | | | | | | | | |
| Rice | 405915 | | | | | | | 115 | 117 | 114 | | | | | | |
| | 408894 | | | | | | | 116 | 117 | 115 | | | | | | |
| | 409338 | | | | | | | 119 | 114 | 107 | | | | | | |
| Pea | 404987 | | | | | | | | | | 117 | 120 | 114 | | | |
| | 407133 | | | | | | | | | | 105 | 117 | 111 | | | |
| | 409386 | | | | | | | | | | 103 | 108 | 97 | | | |
| Soy | 395810 | | | | | | | | | | | | | 115 | 108 | 96 |
| | 405663 | | | | | | | | | | | | | 111 | 106 | 97 |
| | 409547 | | | | | | | | | | | | | 116 | 109 | 100 |

TABLE 20
Original Injections of raw material samples (peak area)

| | Cow Milk-Casein | | | Cow Milk-Whey | | | Rice | | |
|---|---|---|---|---|---|---|---|---|---|
| Sample | Pep1 | Pep2 | Pep3 | Pep1 | Pep2 | Pep3 | Pep1 | Pep2 | Pep3 |
| BCAA:400640 | 116000 | 67800 | 5690 | 1100 | 1830 | 2760 | 509 | 584 | 97 |
| 405984_Milk | 20400000 | 15500000 | 5550000 | 752000 | 1830000 | 1630000 | 0 | 0 | 0 |
| 410058_Milk | 21500000 | 15600000 | 5640000 | 762000 | 1850000 | 1810000 | 0 | 0 | 0 |
| 410057_Milk | 21400000 | 15700000 | 5740000 | 792000 | 2040000 | 1700000 | 0 | 0 | 0 |
| 408894_Rice | 8500 | 5310 | 1050 | 0 | 273 | 476 | 815000 | 3050000 | 86200 |
| 409338_Rice | 8240 | 5570 | 652 | 0 | 365 | 1000 | 1020000 | 6310000 | 113000 |
| 405915_Rice | 9480 | 4710 | 682 | 130 | 476 | 963 | 893000 | 6340000 | 107000 |
| 409386_Pea | 10300 | 6830 | 1040 | 179 | 3110 | 476 | 1180 | 0 | 0 |
| 404987_Pea | 8860 | 6140 | 931 | 195 | 3470 | 446 | 2220 | 0 | 0 |
| 407133_Pea | 9160 | 5680 | 766 | 0 | 3600 | 379 | 1170 | 195 | 0 |
| 405663_Soy | 14600 | 6910 | 1330 | 260 | 2800 | 222 | 352 | 576 | 0 |
| 409547_Soy | 9070 | 5070 | 1230 | 244 | 1990 | 769 | 406 | 303 | 0 |
| 395810_Soy | 9620 | 4600 | 678 | 0 | 2700 | 555 | 0 | 401 | 0 |

| | Pea | | | Soy | | |
|---|---|---|---|---|---|---|
| Sample | Pep1 | Pep2 | Pep3 | Pep1 | Pep2 | Pep3 |
| BCAA:400640 | 552 | 471 | 114 | 698 | 601 | 81 |
| 405984_Milk | 782 | 0 | 0 | 0 | 0 | 0 |
| 410058_Milk | 601 | 0 | 0 | 0 | 0 | 0 |
| 410057_Milk | 471 | 0 | 0 | 0 | 0 | 0 |
| 408894_Rice | 0 | 309 | 0 | 0 | 0 | 0 |
| 409338_Rice | 357 | 0 | 0 | 357 | 0 | 0 |
| 405915_Rice | 390 | 0 | 571 | 0 | 0 | 0 |
| 409386_Pea | 1980000 | 2460000 | 559000 | 4220 | 1960 | 555 |
| 404987_Pea | 2040000 | 2290000 | 527000 | 1420 | 731 | 0 |
| 407133_Pea | 2170000 | 2390000 | 586000 | 0 | 0 | 0 |
| 405663_Soy | 0 | 441 | 0 | 5380000 | 2400000 | 656000 |
| 409547_Soy | 0 | 0 | 384 | 8410000 | 3120000 | 969000 |
| 395810_Soy | 0 | 1090 | 238 | 8610000 | 3270000 | 954000 |

TABLE 21
Reinjection of raw material samples (peak area)

| | Milk-Casein | | | Milk-Whey | | | Rice | | |
|---|---|---|---|---|---|---|---|---|---|
| Sample | Pep1 | Pep2 | Pep3 | Pep1 | Pep2 | Pep3 | Pep1 | Pep2 | Pep3 |
| 405984_Milk_RI | 22100000 | 16900000 | 6440000 | 957000 | 2180000 | 1180000 | 0 | 0 | 0 |
| 410057_Milk_RI | 22900000 | 17100000 | 6580000 | 931000 | 2290000 | 1310000 | 0 | 0 | 0 |
| 410058_Milk_RI | 22300000 | 16600000 | 6490000 | 948000 | 2290000 | 1320000 | 0 | 0 | 0 |
| 405915_Rice_RI | 5350 | 3200 | 731 | 0 | 0 | 1050 | 936000 | 3570000 | 98500 |
| 408894_Rice_RI | 5620 | 2860 | 698 | 0 | 393 | 1390 | 1180000 | 7400000 | 130000 |
| 409338_Rice_RI | 6670 | 3700 | 536 | 0 | 0 | 1230 | 1060000 | 7210000 | 115000 |
| 404987_Pea_RI | 7570 | 4880 | 877 | 0 | 3300 | 0 | 0 | 0 | 0 |
| 407133_Pea_RI | 6210 | 3780 | 698 | 0 | 3390 | 854 | 0 | 0 | 0 |
| 409386_Pea_RI | 6630 | 3100 | 422 | 0 | 4060 | 514 | 0 | 211 | 0 |
| 395810_Soy_RI | 13200 | 5900 | 2050 | 0 | 3070 | 610 | 0 | 0 | 0 |
| 405663_Soy_RI | 8030 | 3210 | 1050 | 0 | 3690 | 292 | 0 | 0 | 0 |
| 409547_Soy_RI | 7270 | 3330 | 1250 | 0 | 3100 | 0 | 0 | 0 | 0 |

| | Pea | | | Soy | | |
|---|---|---|---|---|---|---|
| Sample | Pep1 | Pep2 | Pep3 | Pep1 | Pep2 | Pep3 |
| 405984_Milk_RI | 444 | 0 | 0 | 0 | 0 | 0 |
| 410057_Milk_RI | 703 | 0 | 0 | 0 | 0 | 0 |
| 410058_Milk_RI | 706 | 0 | 0 | 0 | 0 | 0 |
| 405915_Rice_RI | 0 | 0 | 0 | 0 | 0 | 0 |
| 408894_Rice_RI | 385 | 0 | 0 | 0 | 0 | 0 |
| 409338_Rice_RI | 568 | 0 | 544 | 0 | 0 | 0 |
| 404987_Pea_RI | 2310000 | 2950000 | 635000 | 4430 | 1530 | 0 |
| 407133_Pea_RI | 2140000 | 2670000 | 583000 | 2220 | 341 | 0 |
| 409386_Pea_RI | 2230000 | 2590000 | 568000 | 0 | 0 | 0 |
| 395810_Soy_RI | 0 | 0 | 0 | 6170000 | 2580000 | 630000 |
| 405663_Soy_RI | 0 | 1390 | 0 | 9310000 | 3320000 | 941000 |
| 409547_Soy_RI | 0 | 0 | 0 | 10000000 | 3550000 | 957000 |

All references, including publications, patents, and patent applications, cited herein are hereby incorporated by reference to the same extent as if each reference were individually and specifically indicated to be incorporated by reference and were set forth in its entirety herein.

While described in certain embodiments, the present disclosure can be further modified within the spirit and scope of this disclosure. This application is therefore intended to cover any variations, uses, or adaptations of the methods described herein using its general principles. Further, this disclosure is intended to cover such departures from the present disclosure as come within known or customary practice in the art to which this invention pertains and that fall within the limits of the appended claims and their legal equivalents.

### BIBLIOGRAPHIC REFERENCES

1 Haug A, Høstmark A T, Harstad O M, A; Høstmark, A T; Harstad, O M (25 Sep. 2007). “Bovine milk in human nutrition—a review”. Lipids Health Dis 6: 25. doi:10.1186/1476-511X-6-25.
2 McWilliam H., Valentin F., Goujon M., Li W., Narayanasamy M., Martin J., Miyar T. and Lopez R. (2009) Web services at the European Bioinformatics Institute. Nucleic Acids Research 37: W6-W10.

(α-S1-Casein, http://www.ebi.ac.uk/interpro/entry/IPR026999)

(β-Lactoglobulin, http://www.ebi.ac.uk/interpro/entry/IPR002447)
3 Peter R Shewry and Rod Case eds., (1999). Seed Proteins. Kluwer Academic Publishers, Netherlands.
4 O'Kane F E1, Happe R P, Vereijken J M, Gruppen H, van Boekel M A., (2004). Characterization of pea vicilin. 1. Denoting convicilin as the alpha-subunit of the Pisum vicilin family. J Agric Food Chem. 2004 May 19; 52(10):3141-8.
5 Jiang C, Cheng Z, Zhang C, Yu T, Zhong Q, Shen J Q, Huang X, (2014). Proteomic analysis of seed storage proteins in wild rice species of the Oryza genus. Proteome Sci. 2014 Nov. 30; 12(1):51. doi: 10.1186/s12953-014-0051-4.
6 Wolf, W. J. (1972) Purification and properties of the proteins. In: Soybeans: Chemistry and Technology. Vol. 1. Smith, A. K. and Circle, S. J., eds. Avi Publishing Co. Inc., Westport, Conn.
7 Baker, P. R. and Clauser, K. R. http://prospector.ucsf.edu.
8 Madden T. The BLAST Sequence Analysis Tool. 2002 Oct. 9 [Updated 2003 Aug. 13]. In: McEntyre J, Ostell J, editors. The NCBI Handbook [Internet]. Bethesda (Md.): National Center for Biotechnology Information (US); 2002—. Chapter 16. Available from: http://www.ncbi.nlm.nih.gov/books/NBK21097/
9 Protein authenticity, http://www.authentechnologies.com/applications/species-identification/

What is claimed is:

1. A method for determining the presence of one or more proteins in a sample, the method comprising:
enzymatically digesting the sample with a protease activity to generate a plurality of proteolytic peptides;
separating the plurality of proteolytic peptides using liquid chromatography;
performing mass spectrometry on the separated plurality of peptides; and
wherein a protein is present in the sample when three or more target peptides for the protein are present among the proteolytic peptides; and
wherein the target peptides are selected from the groups consisting of SEQ ID NOS:6-8, SEQ ID NOS:9-11, SEQ ID NOS:12-14, SEQ ID NOS:15-17, and SEQ ID NOS:18-20.

2. The method according to claim 1, further comprising adding a known quantity of a standard peptide to the proteolytic peptides.

3. The method according to claim 2, wherein the standard peptide is β-Casomorphin 1-4.

4. The method according to claim 1, wherein the liquid chromatograph is high-performance liquid chromatography.

5. The method according to claim 1, wherein the protease activity is selected from the group consisting of serine proteases, trypsin, hepsin, SCCE, TADG12, TADG14, metalloproteases, PUMP-1, chymotrypsin; cathepsin; pepsin; elastase; pronase; Arg-C; Asp-N; Glu-C; Lys-C; carboxypeptidases A, B, or C; dispase; thermolysin; cysteine proteases, gingipains, and combinations thereof.

6. The method according to claim 1, wherein the protease activity is trypsin.

7. The method according claim 1, wherein the sample is from a food product, a food ingredient, or a nutraceutical product.

8. The method according to claim 1, wherein the one or more proteins are selected from the group consisting of whey, casein, rice, pea, and soy proteins.

9. The method according to claim 1, wherein the one are more proteins are selected form the group consisting of -S1-Casein, β-Lactoglobulin, Vicilin, Glutelin, and Glycinin G1.
